(19) United States
(12) Patent Application Publication
(10) Pub. No.: US 2011/0142961 A1
(43) Pub. Date: Jun. 16, 2011

(54) METHODS AND REAGENTS FOR PREDICTING CLINICAL OUTCOME AND CUSTOMIZING CHEMOTHERAPY IN LUNG CANCER PATIENTS

(75) Inventors: Miguel Tarón Roca, Barcelona (ES); Rafael Rosell Costa, Barcelona (ES)

(73) Assignee: Pangaea Biotech S.A., Barcelona (ES)

(21) Appl. No.: 12/995,196

(22) PCT Filed: May 19, 2009
(86) PCT No.: PCT/EP2009/056045

§ 371 Date: Feb. 14, 2011

(30) Foreign Application Priority Data
May 30, 2008 (EP) 08380167.0

(51) Int. Cl. A61K 33/24; C12Q 1/68; G01N 33/574; A61P 35/00

(52) U.S. Cl. 424/649; 435/6.1; 435/6.12; 435/7.1

(57) ABSTRACT

The invention relates to methods for the prediction of the clinical outcome of a patient suffering from cancer based on the relative expression levels of BRCA1 and RAP80 genes. The invention also relates to anticancer combination therapies comprising a platinum-based chemotherapeutic agent and an inhibitor of RAP80.

### FIELD OF THE INVENTION

The invention relates to the field of pharmacogenomics and, more in particular, to the field of methods for predicting the clinical outcome of patients suffering from small cell lung cancer and, more in particular, advanced small cell lung cancer based on the expression levels of BRCA1 and RAP80. The invention also relates to the filed of cancer therapeutics and, more in particular, to combination therapies for the treatment of cancer based on a platinum-based compounds and an inhibitor of RAP80.

### BACKGROUND OF THE INVENTION

Non-small-cell lung cancer (NSCLC) accounts for approximately 80% of all lung cancers, with 1.2 million new cases worldwide each year. NSCLC resulted in more than one million deaths worldwide in 2001 and is the leading cause of cancer-related mortality in both men and women (31% and 25%, respectively).

NSCLC comprises a group of heterogeneous diseases grouped together because their prognosis and management is roughly identical. However, the following subtypes based on their histology can be identified: (i) squamous cell carcinoma (SCC), accounting for 30% to 40% of NSCLC, also starts in the larger breathing tubes but grows slower meaning that the size of these tumors varies on diagnosis, (ii) adenocarcinoma is the most common subtype of NSCLC, accounting for 50% to 60% of NSCLC, which starts near the gas-exchanging surface of the lung and which includes a subtype, the bronchioalveolar carcinoma, which may have different responses to treatment and (iii) large cell carcinoma is a fast-growing form that grows near the surface of the lung. It is primarily a diagnosis of exclusion, and when more investigation is done, it is usually reclassified to squamous cell carcinoma or adenocarcinoma. Stage grouping of the NSCLC patients in TNM subsets (T=primary tumour; N=regional lymph nodes; M=distant metastases) permits the identification of patient groups with similar prognosis and treatment options. The stages are defined as follows:
Stage I: Cancer is located in only one lung and has not spread to the adjacent lymph nodes or outside the chest.
Stage II: Cancer is located in one lung and may involve lymph nodes on the same side of the chest but does not include lymph nodes in the space between the lungs (the mediastinum) or outside the chest.
Stage IIIA: Cancer is a single tumour or mass that is not invading any adjacent organs and involves one or more lymph nodes away from the tumour, but not outside the chest.
Stage IIIB: Cancer has spread to more than one area in the chest, but not outside the chest.
Stage IV: Cancer has spread, or metastasized, to different sites in the body, which may include the liver, brain or other organs.

The prognosis of advanced NSCLC is dismal. A recent Eastern Cooperative Oncology Group trial of 1155 patients showed no differences among the chemotherapies used: cisplatin/paclitaxel, cisplatin/gemcitabine, cisplatin/docetaxel and carboplatin/paclitaxel. Overall median time to progression was 3,6 months, and median survival was 7,9 months.

The overall five-year survival of patients with NSCLC has remained at less than 15% for the past 20 years. However, the five-year survival varies according to the TNM subset of the patient, being around 60% for pathologic stage IA and IB, 34% for pathological stage IIB (T1-2N1M0, T3N0M0), 13% for stage IIIA (T3N1M0, T1-2-3N2M0), and a low 7% for stage IIIB (T4N0-1-2M0).

In stage I and stage II NSCLC patients, approximately 40% of stage I patients and 66% of stage II NSCLC patients die within five years of surgery, mainly due to the development of distant metastases. This group comprises patients who might derive a notable benefit from adjuvant chemotherapy. Unfortunately, there is at present no reliable clinical predictor of recurrence available.

During the past 30 years medical oncologists have focused to optimize the outcome of cancer patients and it is just now that the new technologies available are allowing to investigate polymorphisms, gene expression levels and gene mutations aimed to predict the outcome and the impact of a given therapy in different groups of cancer patients to tailor chemotherapy.

Although a wealth of data indicates that changes in the level of several gene transcripts can influence the survival expectancy of and the differential chemosensitivity between NSCLC patients with the same TNM subset, at present no predictive genetic markers of chemotherapy response are used for tailoring treatment. To further improve the survival rate in patients with NSCLC, their prognostic classification based on molecular alterations is crucial. Such classification will provide more accurate and useful diagnostic tools and, eventually, more effective therapeutic options.

US20060094021 describes that lung tumors expressing high levels of BRCA1 mRNA are likely to be resistant to platinum-based chemotherapy so that BRCA1 expression levels can be used as a marker for selecting platinum-based chemotherapy in a patient suffering from lung tumor.

WO2006097346 describes that methylation of the 14-3-3 sigma gene promoter region is decreased in tumors which are resistant to cisplatin-based chemotherapy, thus providing for the use of the methylation pattern of the 14-3-3sigma gene as a marker for selecting platinum-based chemotherapy in a patient suffering from lung tumor.

However, all these methods rely on the determination of a single marker. The reliability of the prognostic methods can be increased by simultaneously measuring the expression of several genes whose expression levels correlate with survival among patients with NSCLC. Endoh et al. (J Clin Oncol. 2004, 22:811-9) have described a genomic signature comprising 8 genes which correlates with survival in lung adenocarcinoma. Furthermore, Chen et al. (J Clin Oncol. 2005, 23:953-64) have described a five-gene signature also able to predict survival in NSCLC.

Accordingly, there remains a need in the art for reliable markers which can be used as predictive markers for the outcome of NSCLC patients as well as for customizing chemotherapy in NSCLC patients.

### SUMMARY OF THE INVENTION

The authors of the present invention have found that, unexpectedly, a subgroup of patients suffering from lung cancer and which, based on their low expression levels of the DNA repair protein BRCA1, are sensitive to platinum-based cytotoxic agents, show a worse prognostic when the expression levels of the BRCA1-binding protein RAP80 are higher than reference values. This finding opens the door to new prognostic methods of the clinical outcome of cancer patients by determining the expression levels of BRCA1 and RAP80 and indicates that patients treated with platinum-based chemotherapy may substantially benefit from said chemotherapy when they are simultaneously treated with an inhibitor of RAP80.

Thus, in a first aspect, the invention relates to a method for predicting the clinical outcome of a patient suffering from lung cancer or for selecting a patient for treatment with chemotherapy which comprises determining the expression levels of the RAP80 and BRCA1 genes in a sample from said patient wherein
(i) low levels of BRCA1 and low or intermediate levels of RAP80 or
(ii) high levels of BRCA1 and intermediate levels of RAP80 and are indicative of a positive clinical outcome of said patient or that the patient is not a candidate to be treated with chemotherapy.

In a second aspect, the invention relates to a kit comprising reagents for detecting the expression of BRCA1 and RAP80 genes.

In a third aspect, the invention relates to a composition comprising a platinum-based chemotherapeutic agent and a RAP80 inhibitor.

In a fourth aspect, the invention relates to a composition of the invention for the treatment of cancer.

### BRIEF DESCRIPTION OF THE FIGURES

FIG. 1: Kaplan-Meier curves showing survival for 60 patients in whom RAP80 and Abraxas were analyzed.

FIG. 2: Kaplan-Meier curves showing Survival in NSCLC pts with low BRCA1 levels according to RAP80 mRNA levels.

FIG. 3: Kaplan-Meier curves showing TTP in NSCLC pts with high BRCA1 levels according to RAP80 mRNA levels.

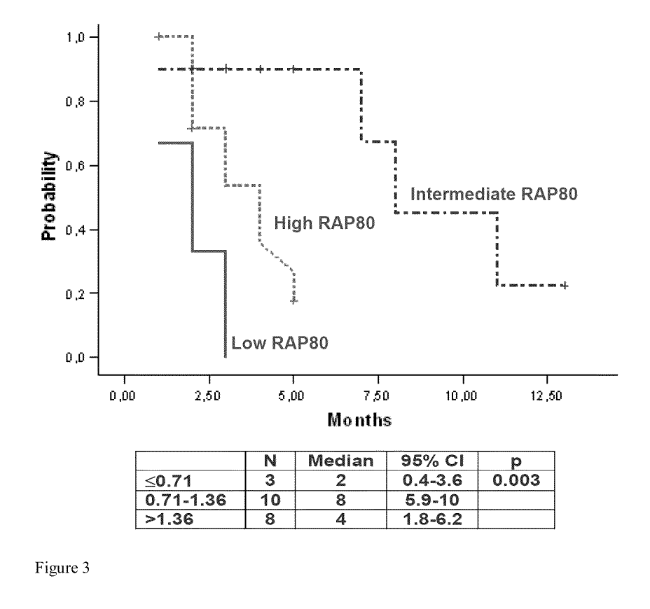

FIG. 4: Survival in patients with high BRCA1 levels according to RAP80 mRNA levels.

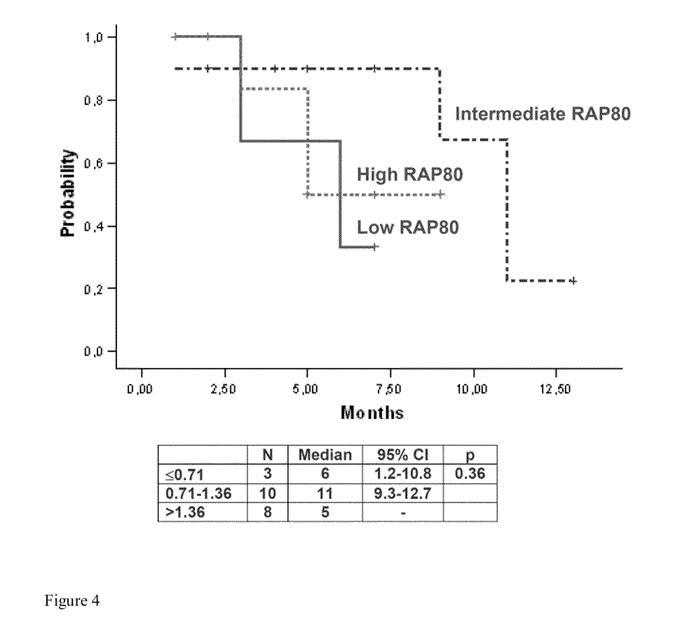

### DETAILED DESCRIPTION OF THE INVENTION

The authors of the present invention have found that, unexpectedly, a subgroup of patients suffering from lung cancer and which, based on their low expression levels of the DNA repair protein BRCA1, are sensitive to platinum-based cytotoxic agents, show a worse prognostic based when the expression levels of the BRCA1 -binding protein RAP80 are higher than the reference values. This finding opens the door to new prognostic methods of the clinical outcome of cancer patients by determining the expression levels of BRCA1 and RAP80 and indicate that patients treated with platinum-based chemotherapy may substantially benefit from when they are simultaneously treated with an inhibitor of RAP80.

Pronostic Methods of the Invention

The authors of the present invention have found that, surprisingly, the combination of expression levels of the BRCA1 and RAP80 genes allows a more accurate prediction of the clinical outcome of a patient suffering from NSCLC that the use of each of genes individually. Thus, in a first aspect, the invention relates to a method for predicting the clinical outcome of a patient suffering from NSCLC cancer or for selecting a patient for treatment with chemotherapy which comprises determining the expression levels of the RAP80 and BRCA1 genes in a sample from said patient wherein
(i) intermediate levels of RAP80 and low levels of BRCA1 or
(ii) low levels of RAP80 and low levels of BRCA1 or
(iii) intermediate levels of RAP80 and high levels of BRCA1 are indicative of a positive clinical outcome of said patient or that the patient is not a candidate to be treated with chemotherapy.

The prediction of the clinical response can be done by using any endpoint measurements used in oncology and known to the skilled practitioner. Useful endpoint parameters to describe the evolution of a disease include, without limitation:
disease-free progression which, as used herein, describes the proportion of patients in complete remission who have had no recurrence of disease during the time period under study
objective response, which, as used in the present invention, describes the proportion of treated people in whom a complete or partial response is observed.
time to progression (TTP), which is a measure of time after the disease is diagnosed or treated until the disease gets worse. The disease is considered to have progressed if both the diameter and volume of the tumour increased by 25 percent or more of the initial measurements, or if a new lesion is evident on CT or MRI scans.
tumor control, which, as used in the present invention, relates to the proportion of treated people in whom complete response, partial response, minor response or stable disease ≧6 months is observed.
disease free survival (DFS) which, as used herein, is defined as the length of time after treatment during which a patient survives with no sign of cancer growth.
six-month progression free survival or PFS6″ rate which, as used herein, relates to the percentage of people wherein free of progression in the first six months after the initiation of the therapy and
median survival (MS) which, as used herein, relates to the time at which half of the patients enrolled in the study are still alive.

In a particular embodiment, prediction of the clinical response is carried out by measuring time to progression and median survival.

The term “lung cancer”, as used herein, relates to any type of lung cancer including squamous cell carcinoma (SCC), adenocarcinoma, bronchioalveolar carcinoma (BAC) or large cell carcinoma (LCC). Moreover, the method of the invention is suitable for the diagnosis of stages I, II, III and IV NSCLC.

The term “sample” as used herein, relates to any sample which can be obtained from the patient. The present method can be applied to any type of biological sample from a patient, such as a biopsy sample, tissue, cell or fluid (serum, saliva, semen, sputum, cerebral spinal fluid (CSF), tears, mucus, sweat, milk, brain extracts and the like). In a particular embodiment, said sample is a tumour tissue sample or portion thereof In a more particular embodiment, said tumor tissue sample is a breast tumor tissue sample from a patient suffering from breast cancer. Said sample can be obtained by conventional methods, e.g., biopsy, by using methods well known to those of ordinary skill in the related medical arts. Methods for obtaining the sample from the biopsy include gross apportioning of a mass, or microdissection or other art-known cell-separation methods. Tumour cells can additionally be obtained from fine needle aspiration cytology. In order to simplify conservation and handling of the samples, these can be formalin-fixed and paraffin-embedded or first frozen and then embedded in a cryosolidifiable medium, such as OCT-Compound, through immersion in a highly cryogenic medium that allows for rapid freeze.

The method of the invention requires determining the expression levels of the BRCA1 and of the RAP80 genes. In a preferred embodiment, the determination of the expression levels of the BRCA1 and RAP80 genes can be carried out by measuring the expression levels of the mRNA encoded by said genes. For this purpose, the biological sample may be treated to physically or mechanically disrupt tissue or cell structure, to release intracellular components into an aqueous or organic solution to prepare nucleic acids for further analysis. The nucleic acids are extracted from the sample by procedures known to the skilled person and commercially available. RNA is then extracted from frozen or fresh samples by any of the methods typical in the art, for example, Sambrook, Fischer and Maniatis, Molecular Cloning, a laboratory manual, (2nd ed.), Cold Spring Harbor Laboratory Press, New York, (1989). Preferably, care is taken to avoid degradation of the RNA during the extraction process.

In a particular embodiment, the expression level is determined using mRNA obtained from a formalin-fixed, paraffin-embedded tissue sample. mRNA may be isolated from an archival pathological sample or biopsy sample which is first deparaffinized. An exemplary deparaffinization method involves washing the paraffinized sample with an organic solvent, such as xylene, for example. Deparaffinized samples can be rehydrated with an aqueous solution of a lower alcohol. Suitable lower alcohols, for example include, methanol, ethanol, propanols, and butanols. Deparaffinized samples may be rehydrated with successive washes with lower alcoholic solutions of decreasing concentration, for example. Alternatively, the sample is simultaneously deparaffinized and rehydrated. The sample is then lysed and RNA is extracted from the sample.

The term “sample” has been previously defined and can be applied to any type of biological sample from a patient. In a particular embodiment, said sample is a tumour tissue sample or portion thereof In a more particular embodiment, said tumor tissue sample is a breast tumor tissue sample from a patient suffering from breast cancer or a formalin embedded breast tissue sample. In a preferred embodiment, the sample is a tumor biopsy.

While all techniques of gene expression profiling (RT-PCR, SAGE, or TaqMan) are suitable for use in performing the foregoing aspects of the invention, the gene mRNA expression levels are often determined by reverse transcription polymerase chain reaction (RT-PCR). The detection can be carried out in individual samples or in tissue microarrays.

In order to normalize the values of mRNA expression among the different samples, it is possible to compare the expression levels of the mRNA of interest in the test samples with the expression of a control RNA. A “Control RNA” as used herein, relates to a RNA whose expression levels do not change or change only in limited amounts in tumor cells with respect to non-tumorigenic cells. Preferably, the control RNA is mRNA derived from housekeeping genes and which code for proteins which are constitutively expressed and carry out essential cellular functions. Preferred housekeeping genes for use in the present invention include β-2-microglobulin, ubiquitin, 18-S ribosomal protein, cyclophilin, GAPDH and actin. In a preferred embodiment, the control RNA is BETA-actin mRNA. In one embodiment relative gene expression quantification is calculated according to the comparative Ct method using β-actin as an endogenous control and commercial RNA controls as calibrators. Final results, are determined according to the formula 2-(ΔCt sample-ΔCt calibrator), where ΔCT values of the calibrator and sample are determined by subtracting the CT value of the target gene from the value of the β-actin gene.

The determination of the level of expression of the BRCA1 gene and RAP80 genes needs to be correlated with the reference values which correspond to the median value of expression levels of BRCA1 and RAP80 measured in a collection of tumor tissue in biopsy samples from cancer patients. Once this median value is established, the level of this marker expressed in tumor tissues from patients can be compared with this median value, and thus be assigned a level of “low,” “normal” or “high”. The collection of samples from which the reference level is derived will preferably be constituted from patient suffering from the same type of cancer. For example, the one described in the examples which is statistically representative was constituted with 60 samples from lung cancer patients. In any case it can contain a different number of samples. The use of a reference value used for determining whether the expression of a gene sample is “increased” or “decreased” corresponds to the median value of expression levels of BRCA1 or RAP80 genes measured in a RNA sample obtained by pooling equal amounts of RNA from each of the tumour samples obtained by biopsy from cancer patients. Once this median value is established, the level of this marker expressed in tumours tissues from patients can be compared with this median value, and thus be assigned a level of “increased” or “decreased”. Due to inter-subject variability (e.g. aspects relating to age, race, etc.) it is very difficult (if not practically impossible) to establish absolute reference values for BRCA1 or RAP80. Thus, in a particular embodiment, the reference values for “increased” or “decreased” BRCA1 and RAP80 expression are determined by calculating percentiles by conventional means involving the testing of a group of samples isolated from normal subjects (i.e. people with no diagnosis of breast cancer) for the expression levels of the BRCA1 gene or of RAP80 gene. The “increased” levels can then be assigned, preferably, to samples wherein expression levels for the BRCA1 or RAP80 genes are equal to or in excess of percentile 50 in the normal population, including, for example, expression levels equal to or in excess to percentile 60 in the normal population, equal to or in excess to percentile 70 in the normal population, equal to or in excess to percentile 80 in the normal population, equal to or in excess to percentile 90 in the normal population, and equal to or in excess to percentile 95 in the normal population.

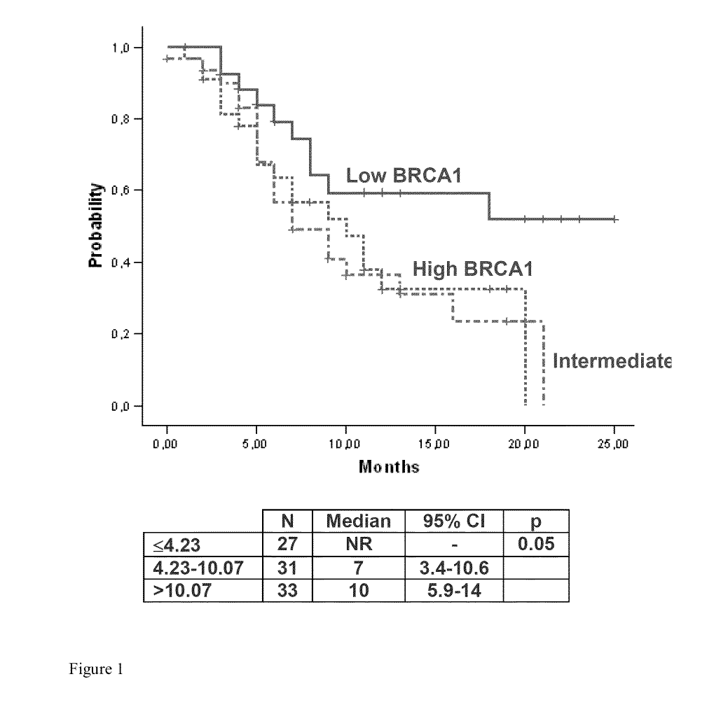

In a preferred embodiment BRCA1 and RAP80 expression values are divided into terciles. As an example, real-time quantitative PCR was used to determine BRCA1 and RAP80 mRNA levels in 60 tumor biopsies from lung cancer patients and divided the gene expression values into terciles. When results were correlated with outcome (survival or TTP), it was observed that patients having intermediate levels of RAP80 and low levels of BRCA1 or low levels of RAP80 and low levels of BRCA1 or intermediate levels of RAP80 and high levels of BRCA1 are characterized by a more positive outcome, i.e. a higher median survival (see FIG. 1).

In another embodiment, the expression levels of the BRCA1 gene and of the RAP80 genes are determined by measuring the expression of the BRCA1 protein and of the RAP80 protein. The determination of the expression levels of the BRCA1 and RAP80 proteins can be carried out by immunological techniques such as e.g. ELISA, Western Blot or immunofluorescence. Western blot is based on the detection of proteins previously resolved by gel electrophoreses under denaturing conditions and immobilized on a membrane, generally nitrocellulose by the incubation with an antibody specific and a developing system (e.g. chemoluminiscent). The analysis by immunofluorescence requires the use of an antibody specific for the target protein for the analysis of the expression and subcellular localization by microscopy. Generally, the cells under study are previously fixed with paraformaldehyde and permeabilised with a non-ionic detergent. ELISA is based on the use of antigens or antibodies labelled with enzymes so that the conjugates formed between the target antigen and the labelled antibody results in the formation of enzymatically-active complexes. Since one of the components (the antigen or the labelled antibody) are immobilised on a support, the antibody-antigen complexes are immobilised on the support and thus, it can be detected by the addition of a substrate which is converted by the enzyme to a product which is detectable by, e.g. spectrophotometry or fluorometry. This technique does not allow the exact localisation of the target protein or the determination of its molecular weight but allows a very specific and highly sensitive detection of the target protein in a variety of biological samples (serum, plasma, tissue homogenates, postnuclear supernatants, ascites and the like). In a preferred embodiment, the BRCA1 protein or the RAP80 protein is detected by immunohistochemistry (IHC) analysis using thin sections of the biological sample immobilised on coated slides. The sections are then deparaffinised, if derived from a paraffinised tissue sample, and treated so as to retrieve the antigen. The detection can be carried out in individual samples or in tissue microarrays.

Any antibody or reagent known to bind with high affinity to the target protein can be used for detecting the amount of target protein. It is preferred nevertheless the use of antibody, for example polyclonal sera, hybridoma supernatants or monoclonal antibodies, antibody fragments, Fv, Fab, Fab′ y F(ab′)2, ScFv, diabodies, triabodies, tetrabodies and humanised antibodies.

In yet another embodiment, the determination of BRCA1 protein and of the RAP80 protein expression levels can be carried out by constructing a tissue microarray (TMA) containing the patient samples assembled, and determining the expression levels of BRCA1 protein and of RAP80 protein by immunohistochemistry techniques. Immunostaining intensity can be evaluated by two different pathologists and scored using uniform and clear cut-off criteria, in order to maintain the reproducibility of the method. Discrepancies can be resolved by simultaneous re-evaluation. Briefly, the result of immunostaining can be recorded as negative expression (0) versus positive expression, and low expression (1+) versus moderate (2+) and high (3+) expression, taking into account the expression in tumoral cells and the specific cut-off for each marker. As a general criterion, the cut-offs were selected in order to facilitate reproducibility, and when possible, to translate biological events.

Kits of the Invention

The methods of the invention require the availability of kits with reagents adequate to determine the expression levels of BRCA1 and RAP80 genes. Thus, in another aspect, the invention relates to a kit comprising reagents for detecting the expression of BRCA1 and RAP80 genes.

The skilled person will appreciate that the reagents of the kits will vary depending on whether the mRNA or the polypeptide encoded by the BRCA1 or RAP80 genes are to be determined. Thus, in preferred embodiments the reagents of the kit are selected from the group of
(i) a probe or primer suitable for the detection and/or amplification of the mRNA encoded by the BRCA1 gene,
(ii) a probe or primer suitable for the detection and/or amplification of the mRNA encoded by the RAP80 gene,
(iii) an antibody or a fragment thereof which binds specifically to the BRCA1 gene product and
(iv) an antibody or a fragment thereof which binds specifically to the RAP80 gene product.

In a preferred embodiment, the kit comprises a first component which is a probe or primer suitable for the detection and/or amplification of the mRNA encoded by the BRCA1 gene and a second component which is a probe or primer suitable for the detection and/or amplification of the mRNA encoded by the RAP80 gene. In another preferred embodiment, the kit comprises an antibody or a fragment thereof which binds specifically to the BRCA1 gene product and an antibody or a fragment thereof which binds specifically to the RAP80 gene product.

Compositions of the Invention

The authors of the present invention have also shown that the survival and time-to-progression of patients showing low levels of BRCA1 and which have been treated with platinum-based compounds is lower when the expression of RAP80 is high than reference values. Without wishing to be bound by any theory, it is believed that the high levels of RAP80 can at least partially compensate for the low levels of BRCA1 so that the DNA lesions induced by cisplatin in the DNA can be repaired by RAP80 thus resulting in that the tumor cells are able to survive to said DNA lesions. This finding opens the door to improving the clinical outcome of patients treated with platinum-based chemotherapy by simultaneous administration with an inhibitor of RAP80. Thus, in another aspect, the invention relates to a composition a platinum-based chemotherapeutic agent and a RAP80 inhibitor.

“Platinum-based compound”, as used herein, is understood as compounds comprising platinum-based chemotherapy drugs which are capable of forming a platinum complex in the cell which are capable of binding and cross-linking DNA, ultimately triggering apoptosis. Platinum-based compounds suitable for use in the present invention includes one or more of carboplatin, cisplatin, oxaliplatin, triplatin tetranitrate, satraplatin and the like.

“RAP80 inhibitors”, as used herein, is understood as compounds which are capable of substantially inhibiting the activity of RAP80. Compounds capable of inhibiting the expression and/or activity of RAP80 include antisense oligonucleotides, ribozymesm siRNAs, inhibitory antibodie, aptamers and spiegelmers. RNAi

In certain embodiments, RNAi may be used to knock down the expression of RAP80 or any component gene necessary for RAP80 function. RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, RNAi may be effected by introduction or expression of relatively short homologous dsRNAs. Indeed, the use of relatively short homologous dsRNAs may have certain advantages as discussed below.

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine residues, which lowers the cost of RNA synthesis and may enhance nuclease resistance of siRNAs in the cell culture medium and within transfected cells (see Elbashir et al., Nature 411: 494-8, 2001). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g., Expedite RNA phosphoramidites and thymidine phosphoramidite (Proligo, Germany)). Synthetic oligonucleotides are preferably deprotected and gel-purified using methods known in the art (see, e.g., Elbashir et al., Genes Dev. 15: 188-200, 2001). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a target nucleic acid, such as, for example, a nucleic acid that hybridizes, under stringent and/or physiological conditions, to the polynucleotide encoding human RAP80.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see, e.g., Jaeger et al., Proc. Natl. Acad. Sci. USA 86: 7706, 1989; and Turner et al., Annu. Rev. Biophys. Biophys. Chem. 17:167, 1988). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerhead ribozyme compositions of the invention.

Several different types of molecules have been used effectively in the RNAi technology. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g., as an antiviral mechanism or in shaping the chromatin structure of a genome. Synthetic siRNAs have been shown to be able to induce RNAi in mammalian cells. This discovery led to a surge in the use of siRNA/RNAi for biomedical research and drug development.

MicroRNA (miRNA) are a related class of gene regulatory small RNAs, typically 21-23 nt in length. They typically differ from siRNA because they are processed from single stranded RNA precursors and show only partially complementary to mRNA targets. Initial studies have indicated that miRNAs regulate gene expression post-transcriptionally at the level of translational inhibition at P-Bodies in the cytoplasm. However, miRNAs may also guide mRNA cleavage similar to siRNAs. This is often the case in plants where the target sites are typically highly complementary to the miRNA. While target sites in plant mRNAs can be found in the 5′UTR, open-reading frames and 3′UTR, in animals, it is the 3′ UTR that is the main target. miRNAs are first transcribed as part of a primary microRNA (pri-miRNA). This is then processed by the Drosha with the help of Pasha/DGCR8 (=Microprocessor complex) into pre-miRNAs. The .about.75 nt pre-miRNA is then exported to the cytoplasm by exportin-5, where it is then diced into 21-23 nt siRNA-like molecules by Dicer. In some cases, multiple miRNAs can be found on the pri-miRNA.

Short hairpin RNA (shRNA) is yet another type of RNA that may be used to effect RNAi. It is a sequence of RNA that makes a tight hairpin turn that can be used to silence gene expression. shRNA is transcribed by RNA polymerase III.

Currently, short-interfering RNAs (siRNAs) and short-hairpin RNAs (shRNAs) are being extensively used to silence various genes to tease out functions carried out by the genes. It is becoming easier to harness RNAi to silence specific genes, owing to the development of libraries of readymade shRNA and siRNA gene-silencing constructs by using a variety of sources. For example, RNAi Codex, which consists of a database of shRNA related information and an associated website, has been developed as a portal for publicly available shRNA resources and is accessible at http://codex.cshl dot org. RNAi Codex currently holds data from the Hannon-Elledge shRNA library and allows the use of biologist-friendly gene names to access information on shRNA constructs that can silence the gene of interest. It is designed to hold user-contributed annotations and publications for each construct, as and when such data become available. Olson et al. (Nucleic Acids Res. 34(Database issue): D153-D157, 2006, incorporated by reference) have provided detailed descriptions about features of RNAi Codex, and have explained the use of the tool. All these information may be used to help design the various siRNA or shRNA targeting RAP80 or other proteins of interest.

Ribozyme

Ribozyme molecules designed to catalytically cleave a target mRNA transcripts can also be used to prevent translation of the subject RAP80 mRNAs and/or expression of RAP80. Ribozymes are enzymatic RNA molecules capable of catalyzing the specific cleavage of RNA. (For a review, see Rossi, Current Biology 4: 469-471, 1994). The mechanism of ribozyme action involves sequence specific hybridization of the ribozyme molecule to complementary target RNA, followed by an endonucleolytic cleavage event. The composition of ribozyme molecules preferably includes one or more sequences complementary to a target mRNA, and the well known catalytic sequence responsible for mRNA cleavage or a functionally equivalent sequence (see, e.g., U.S. Pat. No. 5,093,246, incorporated herein by reference in its entirety).

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach, Nature 334: 585-591, 1988; and see PCT Appln. No. WO89/05852, the contents of which are incorporated herein by reference. Hammerhead ribozyme sequences can be embedded in a stable RNA such as a transfer RNA (tRNA) to increase cleavage efficiency in vivo (Perriman et al., Proc. Natl. Acad. Sci. USA, 92: 6175-79, 1995; de Feyter, and Gaudron, Methods in Molecular Biology, Vol. 74, Chapter 43, “Expressing Ribozymes in Plants,” Edited by Turner, P. C, Humana Press Inc., Totowa, N.J.). In particular, RNA polymerase III-mediated expression of tRNA fusion ribozymes are well known in the art (see, Kawasaki et al., Nature 393: 284-9, 1998; Kuwabara et al., Nature Biotechnol. 16: 961-5, 1998; and Kuwabara et al., Mol. Cell. 2: 617-27, 1998; Koseki et al., J Virol 73: 1868-77, 1999; Kuwabara et al., Proc Natl Acad Sci USA 96: 1886-91, 1999; Tanabe et al., Nature 406: 473-4, 2000). There are typically a number of potential hammerhead ribozyme cleavage sites within a given target CDNA sequence. Preferably the ribozyme is engineered so that the cleavage recognition site is located near the 5′ end of the target mRNA--to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA, such as an mRNA of a sequence represented in any of the human RAP80 proteins. In addition, ribozymes possess highly specific endoribonuclease activity, which autocatalytically cleaves the target sense mRNA. The present invention extends to ribozymes which hybridize to a sense mRNA encoding a target gene such as a therapeutic drug target candidate gene, thereby hybridizing to the sense mRNA and cleaving it, such that it is no longer capable of being translated to synthesize a functional polypeptide product.

The ribozymes used in the compositions of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al., Science 224:574-578, 1984; Zaug et al., Science 231: 470-475, 1986; Zaug et al., Nature 324: 429-433, 1986; published International patent application No. WO88/04300 by University Patents Inc.; Been, et al., Cell 47: 207-216, 1986). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the invention, the gene-targeting portions of the ribozyme or RNAi are substantially the same sequence of at least 5 and preferably 6, 7, 8, 9, 10, 11, 12, 13, 14, 15, 16, 17, 18, 19 or 20 or more contiguous nucleotides of a target nucleic acid, such as a nucleic acid of any of the human RAP80 sequences. In a long target RNA chain, significant numbers of target sites are not accessible to the ribozyme because they are hidden within secondary or tertiary structures (Birikh et al., Eur J Biochem 245: 1-16, 1997). To overcome the problem of target RNA accessibility, computer generated predictions of secondary structure are typically used to identify targets that are most likely to be single-stranded or have an “open” configuration (see Jaeger et al., Methods Enzymol 183: 281-306, 1989). Other approaches utilize a systematic approach to predicting secondary structure which involves assessing a huge number of candidate hybridizing oligonucleotides molecules (see Milner et al., Nat Biotechnol 15: 537-41, 1997; and Patzel and Sczakiel, Nat Biotechnol 16: 64-8, 1998). Additionally, U.S. Pat. No. 6,251,588, the contents of which are hereby incorporated herein, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the RNAi oligonucleotides and ribozymes of the invention.

Antisense Nucleic Acids

A further aspect of the invention relates to the use of the isolated “antisense” nucleic acids to inhibit expression, e.g., by inhibiting transcription and/or translation of a subject RAP80 nucleic acid. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a RAP80 polypeptide. Alternatively, the antisense construct is an oligonucleotide probe, which is generated ex vivo and which, when introduced into the cell causes inhibition of expression by hybridizing with the mRNA and/or genomic sequences of a target nucleic acid. Such oligonucleotide probes are preferably modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al., BioTechniques 6: 958-976, 1988; and Stein et al., Cancer Res 48: 2659-2668, 1988.

With respect to antisense DNA, oligodeoxyribonucleotides derived from the translation initiation site, e.g., between the −10 and +10 regions of the target gene, are preferred. Antisense approaches involve the design of oligonucleotides (either DNA or RNA) that are complementary to mRNA encoding the target polypeptide. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner, Nature 372: 333, 1994). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of mRNA, antisense nucleic acids should be at least six nucleotides in length, and are preferably less that about 100 and more preferably less than about 50, 25, 17 or 10 nucleotides in length.

It is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Results obtained using the antisense oligonucleotide may be compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., Proc. Natl. Acad. Sci. U.S.A. 86: 6553-6556, 1989; Lemaitre et al., Proc. Natl. Acad. Sci. 84: 648-652, 1987; PCT Publication No. WO88/09810) or the blood-brain barrier (see, e.g., PCT Publication No. WO89/10134), hybridization-triggered cleavage agents (see, e.g., Krol et al., BioTechniques 6: 958-976, 1988) or intercalating agents (see, e.g., Zon, Pharm. Res. 5: 539-549, 1988). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl)uracil, 5-carboxymethylaminomethyl-2-thiouridine, 5-carboxymethylaminomethyluracil, dihydrouracil, beta-D-galactosylqueosine, inosine, N6-is opentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine

The antisense oligonucleotide may also comprise at least one modified sugar moiety selected from the group including but not limited to arabinose, 2-fluoroarabinose, xylulose, and hexose. The antisense oligonucleotide can also contain a neutral peptide-like backbone. Such molecules are termned peptide nucleic acid (PNA)-oligomers and are described, e.g., in Perry-O'Keefe et al., Proc. Natl. Acad. Sci. U.S.A. 93: 14670, 1996, and in Eglom et al., Nature 365: 566, 1993. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof

In yet a further embodiment, the antisense oligonucleotide is an alpha-anomeric oligonucleotide. An alpha-anomeric oligonucleotide forms specific double-stranded hybrids with complementary RNA in which, contrary to the usual antiparallel orientation, the strands run parallel to each other (Gautier et al., Nucl. Acids Res. 15: 6625-6641, 1987). The oligonucleotide is a 2′-0-methylribonucleotide (Inoue et al., Nucl. Acids Res. 15: 6131-6148, 1987), or a chimeric RNA-DNA analogue (Inoue et al., FEBS Lett. 215: 327-330, 1987).

While antisense nucleotides complementary to the coding region of a target mRNA sequence can be used, those complementary to the transcribed untranslated region may also be used.

In certain instances, it may be difficult to achieve intracellular concentrations of the antisense sufficient to suppress translation on endogenous mRNAs. Therefore a preferred approach utilizes a recombinant DNA construct in which the antisense oligonucleotide is placed under the control of a strong pol III or pol II promoter. The use of such a construct to transfect target cells will result in the transcription of sufficient amounts of single stranded RNAs that will form complementary base pairs with the endogenous potential drug target transcripts and thereby prevent translation. For example, a vector can be introduced such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, Nature 290: 304-310, 1981), the promoter contained in the 3′ long terminal repeat of Rous sarcoma virus (Yamamoto et al., Cell 22: 787-797, 1980), the herpes thymidine kinase promoter (Wagner et al., Proc. Natl. Acad. Sci. U.S.A. 78: 1441-1445, 1981), the regulatory sequences of the metallothionein gene (Brinster et al, Nature 296: 39-42, 1982), etc. Any type of plasmid, cosmid, YAC or viral vector can be used to prepare the recombinant DNA construct, which can be introduced directly into the tissue site.

Alternatively, target gene expression can be reduced by targeting deoxyribonucleotide sequences complementary to the regulatory region of the gene (i.e., the promoter and/or enhancers) to form triple helical structures that prevent transcription of the gene in target cells in the body (see generally, Helene, Anticancer Drug Des. 6(6): 569-84, 1991; Helene et al., Ann. N.Y. Acad. Sci., 660: 27-36, 1992; and Maher, Bioassays 14(12): 807-15, 1992).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential target sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

Morpholinos

In certain embodiments, the antisense oligonucleotides are morpholino antisenses. Morpholinos are synthetic molecules which are the product of a redesign of natural nucleic acid structure. Usually 25 bases in length, they bind to complementary sequences of RNA by standard nucleic acid base-pairing. Structurally, the difference between Morpholinos and DNA is that while Morpholinos have standard nucleic acid bases, those bases are bound to morpholine rings instead of deoxyribose rings, and linked through phosphorodiamidate groups instead of phosphates. Replacement of anionic phosphates with the uncharged phosphorodiamidate groups eliminates ionization in the usual physiological pH range, so Morpholinos in organisms or cells are uncharged molecules. Morpholinos are not chimeric oligos; the entire backbone of a Morpholino is made from these modified subunits. Morpholinos are most commonly used as single-stranded oligos, though heteroduplexes of a Morpholino strand and a complementary DNA strand may be used in combination with cationic cytosolic delivery reagents.

Unlike many antisense structural types (e.g., phosphorothioates), Morpholinos do not degrade their target RNA molecules. Instead, Morpholinos act by “steric blocking,” binding to a target sequence within an RNA and simply getting in the way of molecules which might otherwise interact with the RNA. Morpholino oligos are often used to investigate the role of a specific mRNA transcript in an embryo, such as eggs or embryos of zebrafish, African clawed frog (Xenopus), chick, and sea urchin, producing morphant embryos. With appropriate cytosolic delivery systems, Morpholinos are effective in cell culture.

Morpholinos are being developed as pharmaceuticals under the name “NeuGenes” by AVI BioPharma Inc. They have been used in mammals ranging from mice to humans and some are currently being tested in clinical trials.

Bound to the 5′-untranslated region of messenger RNA (mRNA), Morpholinos can interfere with progression of the ribosomal initiation complex from the 5′ cap to the start codon. This prevents translation of the coding region of the targeted transcript (called “knocking down” gene expression). Morpholinos provide a convenient means of knocking down expression of the protein and learning how that knockdown changes the cells or organism. Some Morpholinos knock down expression so effectively that after degradation of preexisting proteins the targeted proteins become undetectable by Western blot.

Morpholinos can also interfere with pre-mRNA processing steps, usually by preventing the splice-directing snRNP complexes from binding to their targets at the borders of introns on a strand of pre-RNA. Preventing U1 (at the donor site) or U2/U5 (at the polypyrimidine moiety and acceptor site) from binding can cause modified splicing, commonly leading to exclusions of exons from the mature mRNA. Targeting some splice targets results in intron inclusions, while activation of cryptic splice sites can lead to partial inclusions or exclusions. Targets of U1 1/U12 snRNPs can also be blocked. Splice modification can be conveniently assayed by reverse-transcriptase polymerase chain reaction (RT-PCR) and is seen as a band shift after gel electrophoresis of RT-PCR products.

Morpholinos have also been used to block miRNA activity, ribozyme activity, intronic splice silencers, and splice enhancers. U2 and U12 snRNP functions have been inhibited by Morpholinos. Morpholinos targeted to “slippery” mRNA sequences within protein coding regions can induce translational frameshifts. Activities of Morpholinos against this variety of targets suggest that Morpholinos can be used as a general-purpose tool for blocking interactions of proteins or nucleic acids with mRNA.

DNA Enzyme

A further aspect of the invention relates to the use of DNA enzymes to inhibit expression of target gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

Antisense RNA and DNA, ribozyme, RNAi and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ 0-methyl rather than phosphodiesterase linkages within the oligodeoxyribonucleotide backbone.

Inhibitory Antibodies

Antibodies against an epitope located in RAP80 may effectively block the function of RAP80. Other similar antibodies or fragments thereof may be readily available, or may be readily produced using conventional molecular biology techniques. For example, by using immunogens derived from, for example, RAP80, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., RAP80, or an antigenic fragment thereof, which is capable of eliciting an antibody response). Techniques for conferring immunogenicity on a protein or peptide, include conjugation to carriers or other techniques, are well known in the art. An immunogenic portion of a polypeptide can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immuno-specific for antigenic determinants of RAP80 (or a variant at least 80%, 85%, 90%, 95%, or 98% identical thereto). In certain embodiment, the immunospecific subject antibodies do not substantially cross react with a non-vertebrate (such as yeast) RAP80-related protein. By “not substantially cross react,” it is meant that the antibody has a binding affinity for a non-homologous protein which is at least one order of magnitude, more preferably at least 2 orders of magnitude, and even more preferably at least 3 orders of magnitude less than the binding affinity of the antibody for Rap80.

Following immunization of an animal with an antigenic preparation of a protein, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include for example, the hybridoma technique (originally developed by Kohler and Milstein, Nature 256: 495-497, 1975), the human B cell hybridoma technique (Kozbar et al., Immunology Today 4: 72, 1983), and the EBV-hybridoma technique to produce human monoclonal antibodies (Cole et al., Monoclonal Antibodies and Cancer Therapy, Alan R. Liss, Inc. pp. 77-96, 1985). Hybridoma cells can be screened immunochemically for production of antibodies specifically reactive with the polypeptides of the present invention and monoclonal antibodies isolated from a culture comprising such hybridoma cells. Similarly, hybridoma cells can be screened for the production of antibodies specifically reactive with the polypeptides of the present invention, which also do not substantially cross-reactive with one or more other polypeptides.

The term antibody as used herein is intended to include fragments thereof which are also specifically reactive with one of the subject polypeptides. Antibodies can be fragmented using conventional techniques and the fragments screened for utility in the same manner as described above for whole antibodies. For example, F(ab).sub.2 fragments can be generated by treating antibody with pepsin. The resulting F(ab).sub.2 fragment can be treated to reduce disulfide bridges to produce Fab fragments. The antibody of the present invention is further intended to include bispecific and chimeric molecules having affinity for a protein conferred by at least one CDR region of the antibody.

Both monoclonal and polyclonal antibodies (Ab) have many uses including (1) blocking or antagonizing one or more activities of the subject polypeptide, (2) for detection of the subject proteins (in vitro or in vivo) using standard immunohistochemical/immunocytochemical techniques, (3) for immunodepletion, (4) for immuno-precipitation, and (5) for the immunological screening of cDNA libraries constructed in expression vectors such as λgt11, λgt18-23, λZAP, and λORF8.

The antibodies of the invention can be bound to many different carriers and used to detect the presence of an antigen comprising the polypeptides of the invention. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds.

Another technique that may also result in greater sensitivity consists of coupling the antibodies to low molecular weight haptens. These haptens can then be specifically detected by means of a second reaction. For example, it is common to use such haptens as biotin, which reacts with avidin, or dinitrophenyl, puridoxal, and fluorescein, which can react with specific anti hapten antibodies.

Antibodies, especially monoclonal antibodies or fragments thereof may be cloned, and their coding sequences may be introduced into a target cell by, for example, using any of the expression vectors (viral-based or non-viral vectors) described herein. Such antibodies or fragments, or peptidomimetics thereof may be used to block the activity of RAP80.

The platinum-based chemotherapeutic agent can be used alone or in combination with an antimetabolite. Thus, in a preferred embodiment, the invention relates to a composition according to the invention further comprising an antimetabolite.

“Antimetabolite”, as used herein, is understood as a chemical with a similar structure to a substance (a metabolite) required for normal biochemical reactions, yet different enough to interfere with the normal functions of cells. When the normal function of the cell is cell division, the antimetabolite is useful for the chemotherapy of cancer. Antimetabolites suitable for use in the present invention include folic acid antimetabolites (aminopterin, methotrexate, pemetrexed, Raltitrexed), purine analogs (cladribine, clofarabine, fludarabine, mercaptopurine, pentostatin, thioguanine) and pyrimidine analogs (capecitabine, cytarabine, decitabine, fluorouracil, floxuridine and gemcitabine).

In a preferred embodiment, the antimetabolite is gemcitabine. In another preferred embodiment, the platinum-based compound is cisplatin. In a still more preferred embodiment, the combination of antimetabolite and cytotoxic agent is gemcitabine/cisplatin.

The compositions of the invention may be conveniently formulated for administration with a biologically acceptable medium, such as water, buffered saline, polyol (for example, glycerol, propylene glycol, liquid polyethylene glycol and the like) or suitable mixtures thereof The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the one or more agents. The use of such media for pharmaceutically active substances is known in the art. Except insofar as any conventional media or agent is incompatible with the activity of a particular agent or combination of agents, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations.”

Methods of introduction may also be provided by rechargeable or biodegradable devices. Various slow release polymeric devices have been developed and tested in vivo in recent years for the controlled delivery of drugs, including proteinacious biopharmaceuticals. A variety of biocom-patible polymers (including hydrogels), including both biodegradable and non-degradable polymers, can be used to form an implant for the sustained release of an agent at a particular target site.

Depending on the specific modulators in question, the subject modulators identified using the methods of the present invention may be given orally, parenterally, topically, or rectally. They are of course given by forms suitable for each administration route. For example, they are administered in tablets or capsule form, by injection, inhalation, eye lotion, ointment, suppository, controlled release patch, or infusion.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

One or more agents may be administered to humans and other animals by any suitable route of administration, including orally, nasally, as by, for example, a spray, rectally, intravaginally, parenterally, intracisternally and topically, as by powders, ointments or drops, including buccally and sublingually.

Actual dosage levels of the one or more agents administered in the methods of the present invention may be varied so as to obtain an amount of the active ingredient which is effective to achieve a response in an animal The actual effective amount can be determined by one of skill in the art using routine experimentation and may vary by mode of administration. Further, the effective amount may vary according to a variety of factors include the size, age and gender of the individual being treated. Additionally, the severity of the condition being treated, as well as the presence or absence of other components to the individuals treatment regimen will influence the actual dosage.

The effective amount or dosage level will depend upon a variety of factors including the activity of the particular one or more agents employed, the route of administration, the time of administration, the rate of excretion of the particular agents being employed, the duration of the treatment, other drugs, compounds and/or materials used in combination with the particular agents employed, the age, sex, weight, condition, general health and prior medical history of the animal, and like factors well known in the medical arts.

The one or more modulators identified by the methods of the present invention can be administered as such or in admixtures with pharmaceutically acceptable and/or sterile carriers, and can also be administered in conjunction with other compounds. Such additional compounds may include factors known to influence the proliferation, differentiation or migration of a particular cell. These additional compounds may be administered sequentially to or simultaneously with the compounds being screened by the methods of the present invention. By administering compounds known to influence cell behavior, the invention further contemplates identifying modulators which may not alone be sufficient to influence cell behavior. However, such modulators may be capable of acting additively or synergistically with compounds known to modulate cell behavior.

Modulators screened by the methods of the present invention can be administered alone, or can be administered as a pharmaceutical formulation (composition). The modulators may be formulated for administration in any convenient way for use in human or veterinary medicine. In certain embodiments, the modulators included in the pharmaceutical preparation may be active themselves, or may be a prodrug, e.g., capable of being converted to an active compound in a physiological setting.

Thus, another aspect of the present invention provides pharmaceutically acceptable compositions comprising an effective amount of a RAP80 inhibitor and a platinum-based chemotherapeutic agent, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject agents may be simply dissolved or suspended in sterile water. In certain embodiments, the pharmaceutical preparation is non-pyrogenic, i.e., does not elevate the body temperature of a patient.

The phrase “effective amount” as used herein means that amount of one or more agent, material, or composition comprising one or more agents of the present invention which is effective for producing some desired effect in an animal

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, one or more agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. See, for example, Berge et al., “Pharmaceutical Salts,” J. Pharm. Sci. 66:1-19, 1997).

The pharmaceutically acceptable salts of the modulators include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the one or more modulators may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. See, for example, Berge et al., supra. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate; with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof

It is known that sterols, such as cholesterol, will form complexes with cyclodextrins. Thus, in preferred embodiments, where the inhibitor is a steroidal alkaloid, it may be formulated with cyclodextrins, such as .alpha.-, .beta.- and .gamma.-cyclodextrin, dimethyl-.beta.cyclodextrin and 2-hydroxypropyl-.beta.-cyclo dextrin.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the agents.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle.

Injectable depot forms are made by forming microencapsule matrices of the subject compounds in biodegradable polymers such as polylactide-polyglycolide. Depending on the ratio of agent to polymer, and the nature of the particular polymer employed, the rate of agent release can be controlled. Examples of other biodegradable polymers include poly(orthoesters) and poly(anhydrides). Depot injectable formulations are also prepared by entrapping the agent in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W. H. Freedman and C. O., San Francisco, U.S.A., 1969 or “Livestock Feeds and Feeding” 0 and B books, Corvallis, Oreg., U.S.A., 1977).

The pharmaceutical compositions of the invention are then suitable for the treatment of cancer. Thus, in another aspect, the invention relates to a composition of the invention for the treatment of cancer. In a preferred embodiment, the cancer is lung cancer. In a more preferred embodiment, the lung cancer is advanced lung cancer, in particular, stage IIIB or stage IV lung adenocarcinoma.

Moreover, the pharmaceutical compositions of the invention are particularly suitable for the treatment of patients having low levels of BRCA1 and thus, which respond to platinum-based compounds. Thus, in another embodiment, the composition of the invention is used in patients wherein the expression levels of BRCA1 in a sample from the patient suffering from lung cancer are lower than reference values. Thus, the invention opens the door for personalised treatment of patients with the compositions of the invention based on the expression levels of BRCA1. Measurement of the BRCA1 gene expression levels can be carried out essentially as described previously for the first method of the invention, i.e. by determining BRCA1 mRNA levels or BRCA1 polypeptide levels and using essentially the methods described before. The measurement of the BRCA1 mRNA or polypeptide levels is carried out in a sample of the patient. Samples suitable for carrying out the determination have been mentioned previously. In a preferred embodiment, the sample wherein the expression level of the BRCA1 gene is measured is a sample from a tumour isolated from said patient.

The following methods and examples are to be construed as illustrative and not limitative of the scope of the invention.

### EXAMPLES

Background: A study was carried out wherein treatment of metastatic NSCLC was customized according to the presence of EGFR mutations and BRCA1 mRNA levels.

Methods: 93 patients with lung adenocarcinoma or large-cell carcinoma were screened for EGFR Δ746-750 deletion and L858R mutation and BRCA1 mRNA expression. 15 patients with the L858R mutation in the EGFR gene received erlotinib; 78 patients without the L858R mutation in the EGFR gene received chemotherapy according to BRCA1 levels (low: gemcitabine/cisplatin; intermediate: docetaxel/cisplatin; high: docetaxel alone). In addition, Abraxas and RAP80 mRNA expression were examined in 60/78 patients without the L858R mutation. The features of the patients are collected in Tables I and II. The features of the metastasis in said patients are shown in Tables III and IV. The second line treatment of patients wherein BRCA1 expression levels was measured is shown in Table V.

TABLE I
Characteristics of 89 patients wherein EGFR mutations and BRCA1 expression levels were studied.

| | All patients | | EGFR Mutated | | Low BRCA1 | | Intermediate BRCA1 | | High BRCA1 | | |
|---|---|---|---|---|---|---|---|---|---|---|---|
| | N = 89 | | N = 11 | | N = 32 | | N = 34 | | N = 12 | | p |
| Age | 60 | (40-78) | 62 | (42-75) | 59 | (43-77) | 59 | (40-78) | 63 | (56-75) | 0.24 |
| Sex | | | | | | | | | | | 0.001 |
| Female | 25 | (28.1) | 8 | (72.7) | 9 | (28.1) | 8 | (23.5) | 0 | (0) | |
| Male | 64 | (71.9) | 3 | (27.3) | 23 | (71.9) | 26 | (76.5) | 12 | (100) | |
| Smoker | | | | | | | | | | | 0.03 |
| Current | 29 | (32.6) | 0 | (0) | 10 | (31.3) | 15 | (44.1) | 4 | (33.3) | |
| Never | 15 | (16.9) | 6 | (54.5) | 5 | (15.6) | 4 | (11.8) | 0 | (0) | |
| Ex | 45 | (50.6) | 5 | (45.5) | 17 | (53.1) | 15 | (44.1) | 8 | (66.7) | |
| Race | | | | | | | | | | | 0.61 |
| Caucasian | 88 | (98.9) | 11 | (100) | 31 | (96.9) | 34 | (100) | 12 | (100) | |
| Other | 1 | (1.1) | 0 | (0) | 1 | (3.1) | 0 | (0) | 0 | (0) | |
| ECOG PS | | | | | | | | | | | 0.35 |
| 0 | 37 | (41.6) | 7 | (63.6) | 14 | (43.8) | 10 | (29.4) | 6 | (50) | |
| 1-2 | 52 | (58.4) | 4 | (36.4) | 18 | (56.2) | 24 | (70.6) | 6 | (50) | |
| Histology | | | | | | | | | | | 0.03 |
| Adenocarcinoma | 56 | (62.9) | 7 | (63.6) | 21 | (65.6) | 22 | (64.7) | 6 | (50) | |
| BAG | 4 | (4.5) | 3 | (27.3) | 0 | (0) | 1 | (2.9) | 0 | (0) | |
| Undifferentiated | 11 | (12.4) | 0 | (0) | 4 | (12.5) | 5 | (14.7) | 2 | (16.7) | |
| Other | 18 | (20.2) | 1 | (9.1) | 7 | (21.9) | 6 | (17.6) | 4 | (33.3) | |
| Stage | | | | | | | | | | | 0.45 |
| III | 15 | (16.9) | 1 | (9.1) | 8 | (25) | 4 | (11.8) | 2 | (16.6) | |
| IV | 74 | (83.1) | 10 | (90.9) | 24 | (75) | 30 | (88.2) | 10 | (83.3) | |

TABLE II
Characteristics of 60 patients wherein Abraxas and RAP80 were analysed

| | All patients | | Low | | Intermediate | | High | | |
|---|---|---|---|---|---|---|---|---|---|
| | N = 60 | | N = 23 | | N = 27 | | N = 10 | | p(*) |
| Age | 59 | (40-78) | 58 | (43-75) | 59 | (40-78) | 63 | (56-75) | 0.10 |
| Sex | | | | | | | | | 0.15 |
| Female | 14 | (23.3) | 7 | (30.4) | 7 | (25.9) | 0 | (0) | |
| Male | 46 | (76.7) | 16 | (69.6) | 20 | (74.1) | 10 | (100) | |
| Smoker | | | | | | | | | 0.96 |
| Current | 22 | (36.7) | 9 | (39.1) | 9 | (33.3) | 6 | (60) | |
| Never | 7 | (11.7) | 3 | (13) | 14 | (51.8) | 0 | (0) | |
| Ex | 31 | (51.7) | 11 | (47.8) | 4 | (14.8) | 4 | (40) | |
| Race | | | | | | | | | |
| Caucasian | 60 | (100) | 23 | (100) | 27 | (100) | 10 | (100) | |
| ECOG PS | | | | | | | | | 0.64 |
| 0 | 56 | (93.3) | 21 | (91.3) | 26 | (96.3) | 9 | (90) | |
| 1-2 | 4 | (6.7) | 2 | (8.7) | 1 | (3.7) | 1 | (10) | |
| Histology | | | | | | | | | 0.76 |
| Adenocarcinoma | 40 | (66.7) | 16 | (69.6) | 19 | (70.4) | 5 | (50) | |
| BAC | 1 | (1.7) | 0 | (0) | 1 | (3.7) | 0 | (0) | |
| Undifferentiated | 7 | (11.7) | 2 | (8.7) | 3 | (11.1) | 2 | (20) | |
| Other | 12 | (20) | 5 | (21.7) | 4 | (14.8) | 3 | (30) | |

TABLE III
Metastasis analysis of the NSCLC patients wherein EGFR
mutations and BRCA1 expression levels were measured.

| | All patients | | EGFR Mutated | | Low BRCA1 | | Intermediate BRCA1 | | High BRCA1 | | |
|---|---|---|---|---|---|---|---|---|---|---|---|
| | N = 89 | | N = 11 | | N = 32 | | N = 34 | | N = 12 | | p |
| Adrenal | 6 | (6.7) | 0 | (0) | 3 | (9.4) | 3 | (8.8) | 0 | (0) | 0.52 |
| Pleura | 3 | (3.4) | 1 | (9.1) | 0 | (0) | 2 | (5.9) | 0 | (0) | 0.35 |
| CNS | 13 | (14.6) | 4 | (36.4) | 3 | (9.4) | 3 | (8.8) | 3 | (25) | 0.08 |
| Lung | 24 | (27) | 7 | (63.6) | 7 | (21.9) | 8 | (23.5) | 2 | (16.7) | 0.03 |
| Gland | 8 | (9) | 4 | (36.4) | 2 | (6.3) | 1 | (2.9) | 1 | (8.3) | 0.008 |
| Bone | 8 | (9) | 3 | (27.3) | 3 | (9.4) | 2 | (5.9) | 0 | (0) | 0.1 |
| Skin | 4 | (4.5) | 0 | (0) | 3 | (9.4) | 0 | (0) | 1 | (8.3) | 0.23 |
| Liver | 8 | (9) | 1 | (9.1) | 4 | (12.5) | 2 | (5.9) | 1 | (8.3) | 0.83 |
| Other | 6 | (6.7) | 0 | (0) | 5 | (15.6) | 0 | (0) | 1 | (8.3) | 0.06 |

TABLE IV
Metastasis analysis of the NSCLC patients wherein
Abraxas and RAP80 expression levels were measured.

| | All patients | | Low | | Intermediate | | High | | |
|---|---|---|---|---|---|---|---|---|---|
| | N = 60 | | N = 23 | | N = 27 | | N = 10 | | p |
| Adrenal | 4 | (6.7) | 2 | (8.7) | 2 | (7.4) | 0 | (0) | 0.25 |
| Pleura | 0 | | 0 | | 0 | | 0 | | |
| CNS | 9 | (15) | 3 | (13) | 3 | (11.1) | 3 | (30) | 0.34 |
| Lung | 14 | (23.3) | 5 | (21.7) | 7 | (25.9) | 2 | (20) | 0.91 |
| Gland | 3 | (5) | 1 | (4.3) | 1 | (3.7) | 1 | (10) | 0.72 |
| Bone | 4 | (6.7) | 2 | (8.7) | 2 | (7.4) | 0 | (0) | 0.64 |
| Skin | 3 | (5) | 3 | (13) | 0 | (0) | 0 | (0) | |
| Liver | 6 | (10) | 3 | (13) | 2 | (7.4) | 1 | (10) | 0.80 |
| Other | 4 | (6.7) | 3 | (13) | 0 | (0) | 1 | (10) | 0.16 |

TABLE V
Second line treatment in NSCLC patients wherein
BRCA1 expression levels were measured.

| | All | Low | Intermediate | High |
|---|---|---|---|---|
| | patients | BRCA1 | BRCA1 | BRCA1 |
| Alimta | 1 (9.1) | 1 (20) | 0 | 0 |
| CBCP/Gemcitabine | 1 (9.1) | 0 | 0 | 1 (33.3) |
| CBDP/Docetaxel | 1 (9.1) | 0 | 0 | 1 (33.3) |
| CBDP/Taxotere | 1 (9.1) | 1 (20) | 0 | 0 |
| CDDP/Docetaxel | 1 (9.1) | 1 (20) | 0 | 0 |
| Docetaxel | 3 (27.2) | 2 (400) | 1 (33.3) | 0 |
| Docetaxel/Gemcitabine | 1 (9.1) | 0 | 0 | 1 (33.3) |
| Taxotere/Gemcitabine | 2 (18.2) | 0 | 2 (66.6) | 0 |

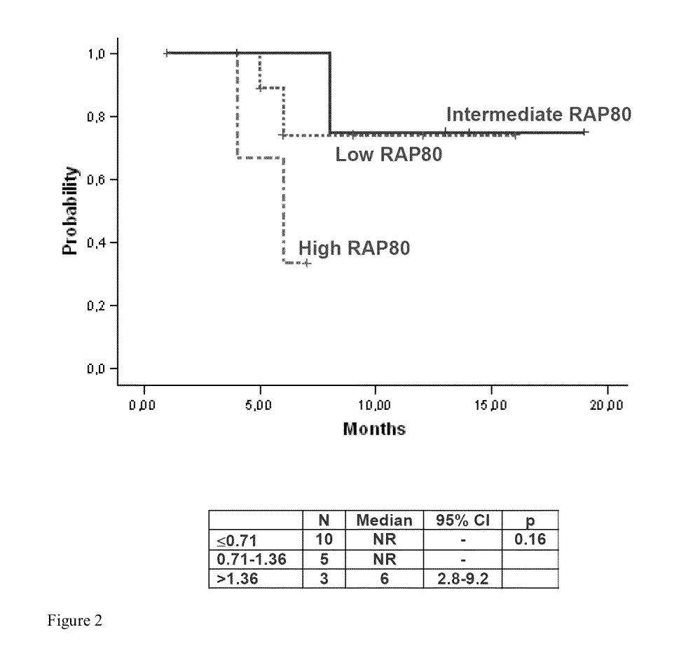

Results: A close correlation was found between BRCA1 and RAP80 levels (p=0.34; P=0.008) and between Abraxas and RAP80 levels (p=0.48; P<0.001). With a median follow-up of 10 months for all 92 patients, median survival was not reached in 11 patients with EGFR mutations. Median survival was not reached in 32 patients with low BRCA1 levels; median survival was 9 months in 34 patients with intermediate BRCA1 levels and 11 months in 12 patients with high BRCA1 levels (P=0.03) (Tables VI and VII and FIG. 1). For the 60 patients in whom Abraxas and RAP80 were analyzed, median survival was not reached in patients with intermediate levels of all 3 genes; median survival was 6 months in patients with low or high levels of all 3 genes and 11 months in other patients (Tables VI and VII and FIG. 2). In the multivariate analysis of survival, only BRCA1 expression emerged as a significant prognostic marker (hazard ratio [HR] for patients with intermediate levels, 4.8 [P=0.02]; HR for patients with high levels, 13.4 [P=0.004]) (HR for performance status 1 =1.8; P=ns).

TABLE VI
Multivariate analysis of survival including BRCA1 by terciles

| | HR | 95% CI | p |
|---|---|---|---|
| ECOG PS | | | |
| 0 | 1 (ref.) | | |
| 1 | 1.8 | 0.3-11.2 | 0.49 |
| 2 | 1.7 | 0.2-18.2 | 0.63 |
| Histology | | | |
| Adenocarcinoma | 1 (ref.) | | |
| BAC | 0 | 0-0 | 0.98 |
| Undifferentiated | 0.09 | 0.01-0.96 | 0.05 |
| Other | 3.1 | 0.7-13.8 | 0.13 |
| No. of Metastases | | | |
| 0-1 | 1 (ref.) | | |
| >1 | 4.9 | 1.2-20.1 | 0.02 |
| Abraxas mRNA levels | | | |
| <0.88 | 1 (ref.) | | |
| 0.88-2.15 | 0.29 | 0.05-1.7 | 0.29 |
| >2.15 | 2.63 | 0.67-10.31 | 0.16 |
| RAP80 mRNA levels | | | |
| <0.71 | 1 (ref.) | | |
| 0.71-1.36 | 0.08 | 0.01-0.48 | 0.005 |
| >1.36 | 0.20 | 0.04-0.88 | 0.03 |
| BRCA1 mRNA levels | | | |
| (terciles) | | | |
| <4.44 | 1 (ref.) | | |
| 4.44-10.9 | 4.8 | 1.29-17.84 | 0.02 |
| >10.9 | 13.4 | 2.34-77.46 | 0.004 |

TABLE VII
Multivariate analysis of survival including BRCA1
by quartiles (used for customizing chemotherapy).

| | HR | 95% CI | p |
|---|---|---|---|
| ECOG PS | | | |
| 0 | 1 (ref.) | | |
| 1 | 2.2 | 0.4-11.9 | 0.36 |
| 2 | 2.7 | 0.3-24.3 | 0.36 |
| Histology | | | |
| Adenocarcinoma | 1 (ref.) | | |
| BAC | 0 | 0-0 | 0.98 |
| Undifferentiated | 0.25 | 0.02-3.22 | 0.28 |
| Other | 7.2 | 1-52.2 | 0.05 |
| No. of Metastases | | | |
| 0-1 | 1 (ref.) | | |
| >1 | 3.6 | 1-12.83 | 0.05 |
| Abraxas mRNA levels | | | |
| <0.88 | 1 (ref.) | | |
| 0.88-2.15 | 0.35 | 0.06-1.99 | 0.24 |
| >2.15 | 1.26 | 0.36-4.42 | 0.71 |
| RAP80 mRNA levels | | | |
| <0.71 | 1 (ref.) | | |
| 0.71-1.36 | 0.11 | 0.11-0.60 | 0.01 |
| >1.36 | 0.27 | 0.06-1.13 | 0.27 |
| BRCA1 mRNA levels | | | |
| (quartiles) | | | |
| Low | 1 (ref.) | | |
| Intermediate | 7.3 | 1.8-29.8 | 0.006 |
| High | 3.9 | 0.4-37 | 0.23 |

Conclusions: Low levels of BRCA1 indicate a favourable outcome to chemotherapy, and BRCA1 may be both a prognostic and predictive biomarker.

1. Method for predicting the clinical outcome of a patient suffering from lung cancer or for selecting a patient for treatment with chemotherapy which comprises determining the expression levels of the RAP80 and BRCA1 genes in a sample from said patient wherein
(i) low levels of BRCA1 and low or intermediate levels of RAP80 or
(ii) high levels of BRCA1 and intermediate levels of RAP80 and are indicative of a positive clinical outcome of said patient or that the patient is not a candidate to be treated with chemotherapy.

2. Method according to claim 1 wherein the expression levels of RAP80 and BRCA1 genes are determined by measuring the amount of the corresponding mRNAs.

3. Method according to claim 1 wherein the expression levels of the RAP80 and BRCA1 genes are determined by measuring the amount of the polypeptides encoded by said genes.

4. Method according to claim 1 wherein the lung cancer is NSCLC.

5. Method according to claim 4 wherein the NSCLC is stage IIIB or IV lung adenocarcinoma.

6. Method according to claim 1 wherein the clinical outcome is predicted as survival or TTP.

7. Method according to claim 1 wherein the sample wherein the expression levels of BRCA1 and RAP80 genes is measured is a sample from the tumor isolated from said patient.

8. A kit comprising reagents for detecting the expression of BRCA1 and RAP80 genes.

9. A kit as defined in claim 8 wherein the reagents are selected from the group consisting of
(i) a probe or primer suitable for the detection and/or amplification of the mRNA encoded by the BRCA1 gene,
(ii) a probe or primer suitable for the detection and/or amplification of the mRNA encoded by the RAP80 gene,
(iii) an antibody or a fragment thereof which binds specifically to the BRCA1 gene product and
(iv) an antibody or a fragment thereof which binds specifically to the RAP80 gene product.

10. A composition comprising a platinum-based chemotherapeutic agent and a RAP80 inhibitor.

11. A composition according to claim 10 further comprising an antimetabolite.

12. (canceled)

13. (canceled)

14. (canceled)

15. (canceled)

16. A method for the treatment of cancer in a subject in need thereof comprising administering to said subject a composition according to claim 10 for the treatment of cancer.

17. A method according to claim 16 wherein the cancer is lung cancer.

18. A method according to claim 17 wherein the lung cancer is advanced lung cancer.

19. A method according to claim 18 wherein the advancer lung cancer is stage IIIB or stage IV lung adenocarcinoma.

20. A method according to claim 16 wherein the patient suffering from lung cancer shows expression levels of BRCA1 in a sample from said patient which are lower than reference value.
